(19) United States
(12) Patent Application Publication
(10) Pub. No.: US 2022/0367014 A1
(43) Pub. Date: Nov. 17, 2022

(54) MULTI-LOCATION, MULTI-ENTITY, MULTI-PURPOSE MEDICAL APPOINTMENT BOOKING SYSTEM WITH THIRD PARTY RESULTS VALIDATION

(71) Applicant: Perry James Brennan, London (GB)

(72) Inventor: Perry James Brennan, London (GB)

(21) Appl. No.: 17/317,992

(22) Filed: May 12, 2021

Related U.S. Application Data
(60) Provisional application No. 63/025,115, filed on May 14, 2020.

(51) Int. Cl. G16H 10/60; G16H 40/20

(52) U.S. Cl. CPC G16H 10/60; G16H 40/20; G06Q 10/10

(57) ABSTRACT

A distance control system for online appointment booking by visiting service providers compares location of a booking applicant with service provider travel distance limitations. When location is beyond the travel distance, a warning message is presented, and alternate service providers are suggested. A method for online document upload and verification of tests results comprises making materials describing patient test results viewable to authorized health professional devices, receiving verification of the materials from the devices, and receiving a dynamic QR code based on the verification and on analysis of materials and determination of patient health status. Managing dynamic codes representing health status comprises issuance of patient health status code based on received input variables, change of code status based on received code updates based on at least one of a pending code expiry, needed medical treatment, and needed patient communication, and comprises issuance of code update based on the changed status.

### REFERENCE TO PRIOR APPLICATION

This application claims priority of the provisional patent application 63/025,115, filed May 14, 2020 entitled MULTI-LOCATION, MULTI-ENTITY, MULTI-PURPOSE MEDICAL APPOINTMENT BOOKING SYSTEM WITH THIRD PARTY RESULTS VALIDATION by Perry James Brennan.

### FIELD OF THE INVENTION

The field of this invention relates generally to the field of health care systems and more particularly toward a multi-functional healthcare system that contains components that can be integrated in part or in whole into a range of existing systems. The components provide scalable enhancements to a range of system types at a chip level, at a software level, or at business levels. One application provides a patient with an accessible platform which enables them to book an appointment, for example with a nearby health care professional (either a hospital or a self-employed doctor, nurse or caregiver) to attain a proper treatment for their health problem. The technical components of the invention may be used for a range of booking, testing or access control systems that use distance, or health status in their processes. Another process invention component further facilitates a user in uploading test results and subsequently verifying or validating the results to obtain the user's health status.

### DESCRIPTION OF THE PRIOR ART

A prominent reason that health suffers is stress. With stress comes health related problems including depression, anxiety, high blood pressure, and heart disease. All the aforementioned diseases are very dangerous and require medical attention. People with these health-related problems are often required to visit doctors to obtain proper treatment to treat the disease. An individual may need to make an appointment before receiving care.

Making an appointment with a healthcare provider can be difficult because a person may need to visit a hospital or clinic and be burdened with standing in a queue. Receiving healthcare may involve a human operator to manage the final stages of a home visit. Hospitals and clinics have started to schedule appointments via telephone calls. People are facing issues in booking appointments because the called line may be busy. Hospitals and clinics have dealt with appointment problems by booking appointments online by having patients register on a website via a user-interface. However, at the time of booking the appointments, people may be unaware of how far the hospital or clinic is from their location and they may be forced to travel more than usual. Consequently, they may be late or miss their appointments. Currently, there is no system that aids patients or customers in booking of home visit appointments and calculates the distance before confirming a booking at the nearest healthcare facility from their home.

Websites or applications are available that are focused on helping customers in booking the appointment of health or other services appointments for home visiting services. If a patient or customer wishes to go to a particular healthcare professional, then a range of webpages or user-interfaces or even hardware devices such as PDAs, mobile devices, and specialist machines may be in use. However, there is no system that allows an individual to join the same user platform as used by the companies.

Another problem faced by people while booking an appointment regards their health status based on recently taken tests and if that person is safe to undertake certain activities or for others nearby that they are safe. To overcome this problem, a solution is provided herein for a declaration of health status. Any interested parties can feel safe that the health status of a customer is certified by a health professional. Governments, companies, airlines etc., hospitals can feel confident that a customer is safe. A customer can upload an image of the test result with whom the patient had earlier consulted. However, for verification of the uploaded test result the patient may be required to open an account with ID and address checks as well as test documentation check.

In recent times of COVID-19, the test reports and health status of a customer may need to be verified at the earliest by the health professional in order to provide an accurate Health Status. Nowadays, if a person wishes to visit another country, then they may need to carry a negative COVID report or Vaccination Certificate or else they may not be allowed to travel. People may be required to verify their reports from the health professional in order to travel. However, existing systems that help certify a person's health status are not in existence. They are fixed status declarations with predetermined parameters. Dynamic status adjustments for Codes or QR codes for an instant verification or validation of Health Status is not previously implemented. Health status can change from anywhere and anytime with a dynamic set of variables influencing the health status over a variable amount of time.

While the prior art includes online booking platforms, none provides a platform that connects a user to a nearby health professional to save time and travel of the user and health professional. Furthermore, online validation of reports is taught by the prior art but such items of prior art do not accept a snapshot or scanned copy of the health-related documents for validation. Moreover, there are many web applications that are being run on static codes that once established cannot be changed.

Therefore, there is a need for systems and methods that accept an image or scanned copy of a health-related document online along while providing a platform that connects the user with the health professional. Additionally, dynamic codes are generated in systems provided herein so that changes in the user's health status are displayed. Systems and methods provided herein overcome disadvantages and limitations associated with the prior art and provide a more satisfactory solution to the problems described above.

### SUMMARY OF THE INVENTION

In an embodiment, a distance control system for online appointment booking by visiting service providers is provided. The system comprises a computer and at least one application executing thereon that receives a message from a booking applicant requesting a home visit appointment with a first service provider. The system also checks location of the booking applicant and checks current parameters set by the first service provider for distance willing to travel. Based on a determination that the location is beyond the distance willing to travel, the system presents a warning message to the booking applicant that the location is beyond the distance. The system also suggests at least a second service provider to the booking applicant. The computer books an appointment for a requesting second booking applicant with the first service provider upon determining that location of the second booking applicant is within the distance willing to travel.

In another embodiment, method is provided for online document upload and verification of tests results. The method comprises a computer receiving materials comprising at least images and reports of medical test results from at least one of a patient and a third-party. The method further comprises the computer making the materials viewable to an authorized health professional device and receiving verification of the materials from the device. The method further comprises the computer receiving a dynamic QR code, the code based on the verification and further based on analysis of the materials and determination of a health status of the patient.

In another embodiment, a system for managing dynamic codes representing health status is provided. The system comprises a computer and application executing thereon that issues a health status code for a patient based on a first received set of input variables, the code represented as a QR code. The system also changes a status of the code based on a received update to the code, the received update based on at least one of a pending expiry of the code, a need for the patient to receive a medical treatment, and a need for the patient to initiate a communication. The system also issues an update to the code based on the changed status.

### BRIEF DESCRIPTION OF THE DRAWINGS

For a better understanding of the present invention, reference is to be made to the accompanying drawings. It is to be understood that the present invention is not limited to the precise arrangement shown in the drawings.

FIG. 1 illustrates a method of distance calculation for outcall visits in accordance with booking of an appointment.

FIG. 2 illustrates a method of third-party test results verification/inclusion in accordance with booking of an appointment.

FIG. 3 illustrates a process flow of a third-party update status of verification in the form of a QR scan code in accordance with an embodiment of the present disclosure.

FIG. 4 illustrates a system allowing customers to book individuals and companies and other entities, with one or more locations, and with incall or outcall appointments controlled by distance and other parameters set by the entities in accordance with an embodiment of the present disclosure.

FIG. 5 illustrates a method flow diagram of the multi-functional healthcare system in accordance with online booking of an appointment.

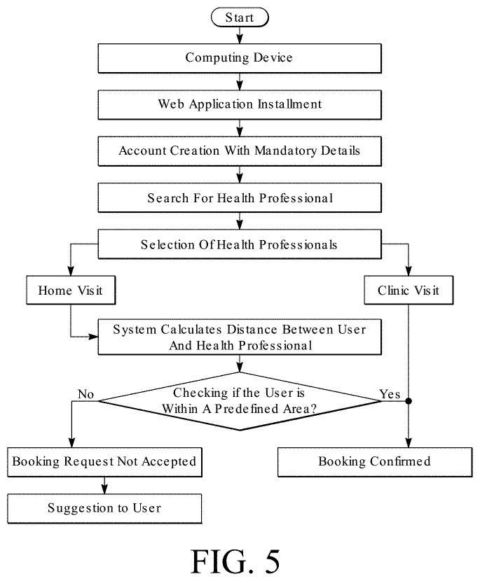

FIG. 6 illustrates a method flow diagram of the multi-functional healthcare system in accordance with uploading and validation of documents;

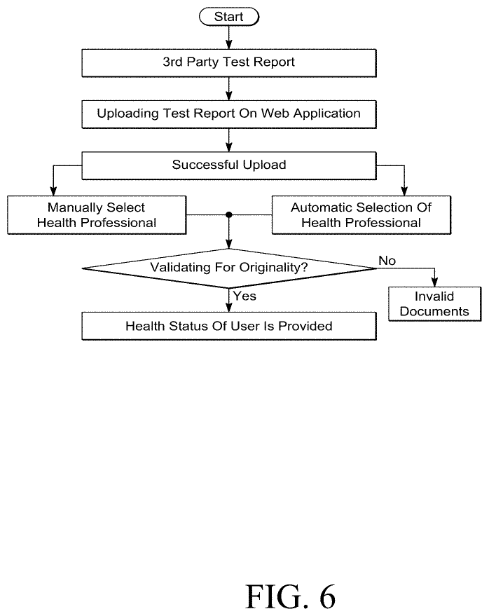

FIG. 7 illustrates an access control flow diagram of the multi-functional healthcare system; and

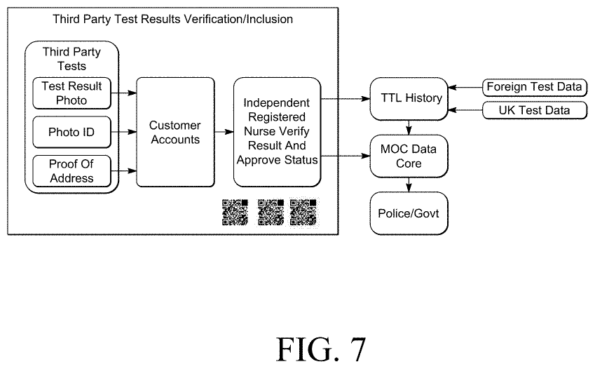

FIG. 8 illustrates a perspective view of the multi-functional healthcare system that shows wireless connectivity between the user and health professional.

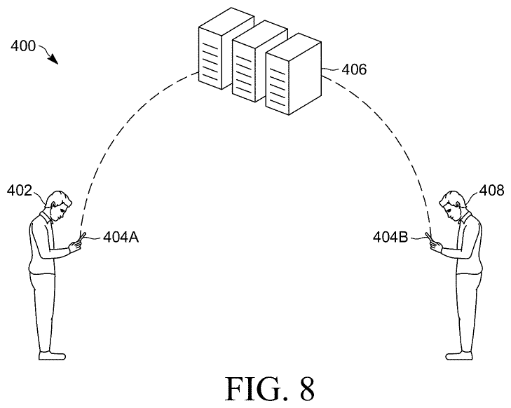

### DETAILED DESCRIPTION OF A PREFERRED EMBODIMENT

Turning to the drawings, the preferred embodiment is illustrated and described by reference characters that denote similar elements throughout the several views of the instant invention.

The preferred embodiment of the instant invention enables a booking process for individuals as well as organizations. The benefit is that health professionals can now receive bookings as individuals not just as companies. The benefit for customers is that they can now choose between companies and individuals on the same booking system. And there can be multiple locations for each entity. Other systems do not have this combined function to allow self-employed individuals to join the system.

This complete booking platform for both individuals and companies reflects a different structure to be able to support each type of entity. That leads to individual profile pages for each entity and company profiles and individual profiles so user can compare with reviews and content to support their choices with patient reviews on the entity so customers can make choices about which entity to obtain a service from. Registration and qualifications are all that are checked with the system and the reviews from customers and the content added to the system by the individuals and companies is what the customers use to choose which entity they will make a booking with.

The subject invention offers a single app or website to the patient for selecting the medical services options from multiple healthcare or organizations at single or multiple locations, where the patient can book appointments across multiple organizations and multiple locations and types of medical tests and vaccinations.

Self-employed single people, health professionals, as well as companies can connect into the system.

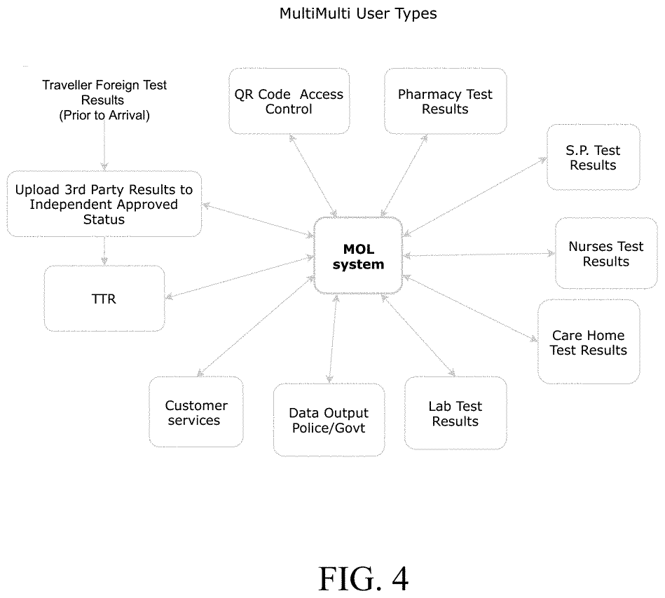

The system allows customers to book individuals and companies and other entities, with one or more locations, and with in-call or outcall appointments controlled by distance and other parameters set by the entities. The system is further illustrated in FIG. 4.

Systems and methods described herein combine computer servers, portable devices and chip architecture programming to enhance multi-purpose appointment booking and event management systems. Systems and methods provided herein are directed to at least improvement of some systems to be faster and more efficient and also to solve appointment booking problems. Attributes of the present disclosure may improve on current offerings and provide solutions to more recent problems. Method steps provided herein may be implemented at an architecture hardware chip level or at an application level.

When a customer requests to make a booking for a service provider to visit, a distance calculation system and method for outcall visits calculates distance based on customer location and variable parameters set by the service provider for outcall distance limits. The system approves and/or rejects booking and suggests the customer choose another location or service provider for the booking and possibly also makes suggestions regarding at least other locations or service providers. The combination of outcall and in-call visits based on location information of the patient and the distance control is of use to customers and service providers to be warned that they are too distant from each other, and the service provider will not be willing to travel that far to provider their requested service. Outcalls comprise visits by service provider to customer location. In-calls comprise visits by customer to service provider location.

After checking current parameters set by the service provider for distance willing to travel. And checking the location of the booking applicant. If the applicant is outside of the distance parameter set by the Service Provider, a warning message is presented to the applicant of the booking that they are too far and a suggestion on alternative options is provided. If the applicant is within the current distance parameter set by the Service Provider, then an appointment may be made

A chip level architecture implementation it will be faster, especially when using existing GPS or Geo Location Chip designs that use their existing calculation functionality for geo locations. Adding distance control and suggestions is now possible due to smart phones with geo location chips and applications and server mapping systems.

Current systems need additional steps of human involvement to verify and validate distances for online booking processes. Systems and methods provided herein yield a more efficient process, faster and more efficient than manual processes that validate distances for bookings.

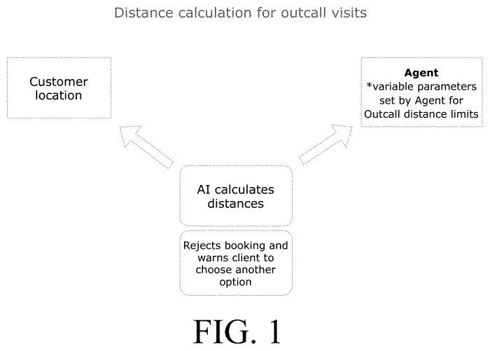

Use of distance control protocols has a plurality of applications. Inserting the protocols into chips and application-level systems for use in large scale use has application in many areas. The Distance Control method provided herein can be used for many types of services that require home or office visits. Nurses, doctors, caregivers, hairdressers, service engineers, and plumbers all can be helped by this distance controller process for online bookings. FIG. 1 is a block diagram of distance calculation for outcall visits.

Current systems require gatherers and users of third party tests and vaccination evidence to have a digital connection and to be preapproved for connection into each other's data systems. Organizations need to be connected to each other for pre-approved processes. The data of these connected systems is shared and reviewed. Most organizations do not have legal jurisdiction across borders to be able to share data and connect to each other's systems.

The method does not need digital authorized connections. The method provides an image based Third-Party test and vaccination document verification system. Users upload their test result documents to the servers. Trained professionals analyze the documents. The method comprises: Test result documents, images of the documents, and scans of the actual documents can be uploaded by the customer to their account for approval. There the method provides for receiving images of documents of third-party test results, medical reports, photographs, and other information from a third-party or from the patient/user. The system collects and stores photos or scans of medical reports regarding the patient in their account.

Then an authorized health professional reviews the uploaded materials. The materials are then visually or digitally checked for fraud or authenticity and validation occurs. Sharing of test results data is not the same as viewing the actual report document. The uploaded materials are verified by an authorized health professional who issues an opinion on the health status from at least that review. To judge if a document is false or real can be done at least by viewing it. Digital data is easily manipulated. Physical documents allow for additional validations. Health care professionals view photos or scans of physical documents.

The authorized health professional can have an account. The user can select the authorized health professional and the system can select the authorized health professional. Digital checking assistance can be done by connecting to some database with known or suspected fraud or fake or other issues. Documents may be compared to assist the authorized health professional to grant the health status.

The health professional forms an opinion about the document, not about the test results but about the document's authentic character and the information contained in it and whether that information makes sense. Databases of labs can assist in such reviews, but the status of the individual can be validated by the health professional and published in the system for use by the customer and the system. The opinion of the status translates into a resulting health status in the form of a machine and human readable code or image, wherein the machine-readable codes are for example dynamic QR codes. They could be any type of codes or image or video but in this example, it is a QR code.

The opinion of the health professional translates into the form of a machine and human readable code or image that are further displayed to the user on their screen. Machine-readable codes are of many types like image, video, shape, letter and many more but the machine-readable code used herein is a QR code. The QR code herein is generated upon validation of the report. The QR code herein shows the opinion of the health professional towards the user's verified report. The code thus formed is further used by the user or lab or the system for access control purposes. The health status provided by the health professional is also used by the government health service, police officials for legal requirements. The government of other country may access the health status of the user, if the user wishes to travel abroad. This saves the time and effort of the user earlier required in obtaining a valid health report for travelling. This facility is availed by the user mostly before embarking their travel.

The online organization receives the third-party test results/medical information from a third-party or from patient by upload or some kind of transmission method. The patient is uploading the third-party test results. Not the third party. The third party is a lab from another country or another lab in the same country. The third party may be a foreign health professional from outside of the system provided herein. The third party creates the test results. The customer/patient is not the third party. The system then can share this customer medical information. After the medical information is added to the system, the test results are verified or approved by an authorized health professional.

The health care professional validates the document and the test data contained in the document. But the health care professional was not present when the test was conducted so the health care professional cannot validate the test itself. The health care professional can only validate if the data of the test seems reasonable. The health care professional validates the tests or medical information that is added into the system. The health professional is then able to issue a status or opinion on the third-party medical data that it is valid and the patient therefore many have a certain health status as a result. The result can take various forms, for example a letter, a code, a symbol, a sign, or a text form. This status can then be used by the customer, a third party, or the system for access control purposes. Further, a government health service, or police may access a health status and its related documents for legal requirements.

Patients' photos and scans can now be added to online systems of test results. Test result documents can be validated from most labs without the need to establish digital data connections between agreeing parties. Individual patients can upload their documents of their test results directly for validation by a health professional. This can be done before travel is embarked upon. Health status can be validated before arrival or at arrival to avoid delays.

Images of documents uploaded by the patient to the systems server get reviewed by the health professional. The health professional may be assisted by data systems.

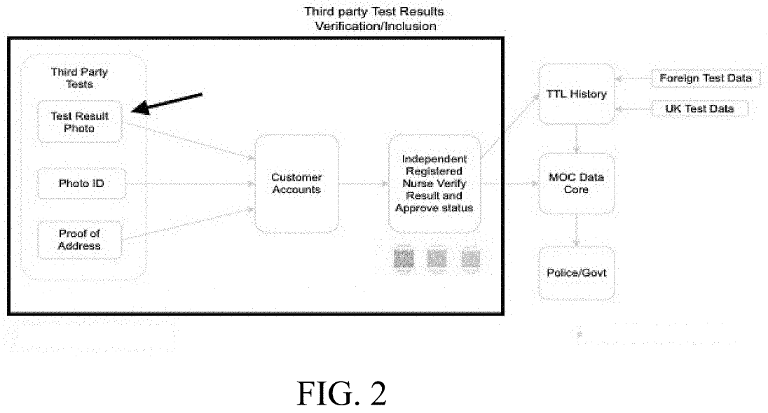

The system and method of this claim may be scalable and be integrated in part or whole into locally or globally configured small or large systems. Digital image storage systems are a possible path for a rapid storage and delivery of test documents to the reviewer. Digital scanning of the documents to assist the reviewer can also help validate a document and the resulting health status. FIG. 2 illustrates verification and inclusion of third-party test results.

Existing codes which provide a health status are static. Static means that when they are given, they have a predefined expiry date or no expiry date. They are a single use code set at the time of creation. That is the end of the code.

Systems and method described herein provide a code status that is updated multiple times during its life cycle. An administrator of the site activates an update or submit button when the input variables are changed. All codes are updated as they are connected into the system. Codes are not an image disconnected to the system. They are still live connected parts of the system and are located as part of a customer's account. This is dynamic. The updates can take place in real time or can be delayed. The updates to the code change the status of the code. For example, an update that affects the expiry date is variable. The expiry date can be changed according to the input parameter which controls the expiry date of the code.

In addition, there can be a warning of the impending expiry date. The code can provide a visual or audio warning to warn the user of the pending expiry. A visual warning can be some visual change in the code. For example, flashing or blinking or color or any visual change to the code. An audio signal or warning may also be used. A visual warning could be a range of options including but not limited to change shape or color for example to indicate its pending expiry. The value of the warning could be many reasons but could include: The warning may be valuable in several ways including providing a reminder to get retested or have a vaccination booster or simply to contact the company about any number of issues.

Another benefit of the input control of a codes status is that it could be cancelled immediately. This might be useful, for example, if there has been a change in government policy regarding status. Or if the user of the code is abusing the use of the code and authorities or the company decide the code of that individual user should be cancelled.

If a patient is abusing or misusing their QR code health status, it may be cancelled according to government or court or police requests. Other QR codes do not offer this process option.

Current systems provide preset status results with predefined outcomes at the moment of the status creation. This method of making a health status is dynamic. Adjustable parameters may be applied to the health status after an initial status has been established. A health status can therefore roll on and on morphing into different states depending on the variables applied to it.

Cameras, servers, chips, and other systems. Artificial intelligence can benefit from a dynamic health status. Scanners, hardware devices, access control points, government and health Policy makers all have connectivity to this dynamic health status method.

A dynamic health status can be used in the smallest situation such as a hair salon or the industrial scale environment such as a border control or police environment.

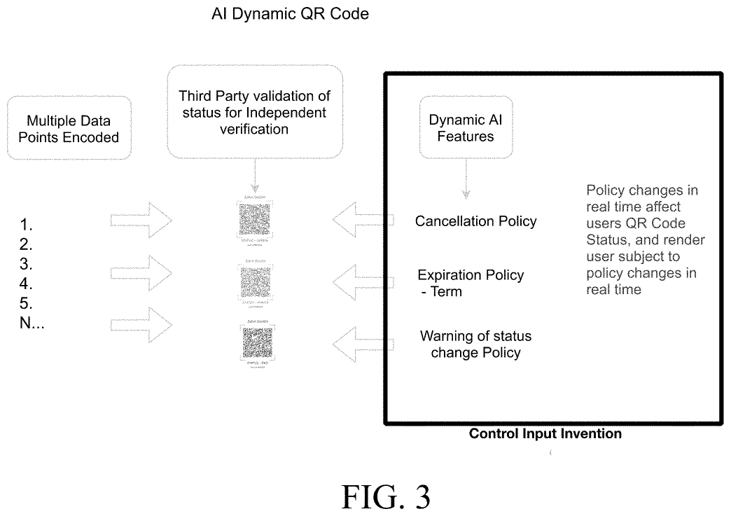

The process flow of FIG. 3 shows a third-party update status of verification in the form of a QR scan code. The QR code included dynamic AI features with terms of different policies (i.e., Extending the expiry date, Cancellation policy, and expiration policy).

The QR Code is not just live virus testing. It's also for antibody results and vaccination status too. A variety of inputs can be used to establish a health status. And a variety of further inputs can be used to make it dynamic. As experts learn about the longevity of antibodies, the QR Code status can be extended or shortened as health authorities or government policy is developed based on new knowledge about the disease or any new diseases or variants. Policy may also be developed based on greater understanding about various health conditions such as sensitivity for users' blood type, age, race, or genetic or health conditions. There could be many reasons to vary an individual or a group of individuals' health status.

If a patient is abusing or misusing their QR code health status, it may be cancelled according to government or court or police requests. Other QR codes do not offer this process option.

If health authorities find that there is mass fraud on any testing or errors the status may be rescinded, and the patient advised to get a new test.

In some cases, the system allows for test results from other third parties and their systems to be added into the Multi Test Results and Booking System.

The health status and resulting access control can be dynamically modified or updated after the initial status has been issued. In the case of the symbol or QR code this can be modified to reflect the change in status or access control aspects desired by the system.

If health authorities find that there is mass fraud on any testing or errors, the status may be rescinded, and the patient advised to get a new test. This status can also then be used by third party or the system for access control purposes. Further, a government health service police may access certain data types if authorized. Anyone not connected to the system is a third party. For example, a hair salon can use the status to allow a customer to enter their shop. Or any company or household can view the status or scan it and decided to allow that person to enter a certain location.

“Code Dynamic Control Example Rules” The invention allows for the existing QR codes to be altered as needed over time. The existing Health Status of the Code provided changes according to input changes in the control of the code status. Adjustments to the input parameters can include but not be limited to some examples below. These updates to the code can be done on an individual or group basis. Status can be made to vary depending on the type of health test or status provided or service or health condition of the individual or group of individuals.

The invention illustratively disclosed herein suitably may be practiced in the absence of any element which is not specifically disclosed herein.

The discussion included in this patent is intended to serve as a basic description. The reader should be aware that the specific discussion may not explicitly describe all embodiments possible and alternatives are implicit. Also, this discussion may not fully explain the generic nature of the invention and may not explicitly show how each feature or element can actually be representative or equivalent elements. Again, these are implicitly included in this disclosure. Where the invention is described in device-oriented terminology, each element of the device implicitly performs a function. It should also be understood that a variety of changes may be made without departing from the essence of the invention. Such changes are also implicitly included in the description. These changes still fall within the scope of this invention.

Further, each of the various elements of the invention and claims may also be achieved in a variety of manners. This disclosure should be understood to encompass each such variation, be it a variation of any apparatus embodiment, a method embodiment, or even merely a variation of any element of these. Particularly, it should be understood that as the disclosure relates to elements of the invention, the words for each element may be expressed by equivalent apparatus terms even if only the function or result is the same. Such equivalent, broader, or even more generic terms should be considered to be encompassed in the description of each element or action. Such terms can be substituted where desired to make explicit the implicitly broad coverage to which this invention is entitled. It should be understood that all actions may be expressed as a means for taking that action or as an element which causes that action. Similarly, each physical element disclosed should be understood to encompass a disclosure of the action which that physical element facilitates. Such changes and alternative terms are to be understood to be explicitly included in the description.

What is claimed is:

1. A distance control system for online appointment booking by visiting service providers, comprising:
a computer and at least one application executing thereon that:
receives a message from a booking applicant requesting a home visit appointment with a first service provider,
checks location of the booking applicant,
checks current parameters set by the first service provider for distance willing to travel,
based on a determination that the location is beyond the distance willing to travel, presents a warning message to the booking applicant that the location is beyond the distance, and
suggests at least a second service provider to the booking applicant,
wherein the computer books an appointment for a requesting second booking applicant with the first service provider upon determining that location of the second booking applicant is within the distance willing to travel.

2. A method for online document upload and verification of tests results, comprising:
a computer receiving materials comprising at least images and reports of medical test results from at least one of a patient and a third-party;
the computer making the materials viewable to an authorized health professional device;
the computer receiving verification of the materials from the device; and
the computer receiving a dynamic QR code, the code based on the verification and further based on analysis of the materials and determination of a health status of the patient.

3. A system for managing dynamic codes representing health status, comprising:
a computer and application executing thereon that:
issues a health status code for a patient based on a first received set of input variables, the code represented as a QR code;
changes a status of the code based on a received update to the code, the received update based on at least one of a pending expiry of the code, a need for the patient to receive a medical treatment, and a need for the patient to initiate a communication, and
issues an update to the code based on the changed status.
